(19) United States
(12) Patent Application Publication
(10) Pub. No.: US 2016/0175569 A1
(43) Pub. Date: Jun. 23, 2016

(54) DEVICE FOR TREATING VASCULAR OCCLUSION

(71) Applicant: Richard R. Heuser, Phoenix, AZ (US)

(72) Inventor: Richard R. Heuser, Phoenix, AZ (US)

(21) Appl. No.: 14/579,907

(22) Filed: Dec. 22, 2014

(51) Int. Cl. A61M 25/10

(52) U.S. Cl. CPC A61M 25/10181; A61M 2025/1063

(57) ABSTRACT

Disclosed herein are extension members for use with a standard guide catheter, which extension members provide backup support against forces that would otherwise tend to dislodge the guide catheter from the branch artery during interventional cardiology devices. During use, the extension member may be deeply seated in a branch artery that branches off from a main artery when extended through the lumen of the guide catheter and beyond the distal end of the guide catheter. An expandable balloon disposed adjacent the distal tip of the extension member provides additional stabilization and backup support. Also disclosed are methods of using the extension members.

### TECHNICAL FIELD

Embodiments relate to devices in interventional cardiology procedures, such as methods and apparatus for treating stenosis and vascular occlusions.

### BACKGROUND

In coronary artery disease, the coronary arteries are narrowed or occluded by atherosclerotic plaques or other lesions that may totally obstruct or dramatically narrow the lumen of the artery. In order to diagnose and treat obstructive coronary artery disease, it is commonly necessary to pass an instrument, such as a guidewire, stent, or balloon catheter through an occlusion or stenosis. For example, in treating a stenosis, a guide catheter may be inserted through the aorta and into the ostium of the coronary artery with the aid of a guidewire. The guide catheter may be seated into the opening or ostium of the artery to be treated, and a guidewire or other instrument may be passed through the lumen of the guide catheter and inserted into the artery near the occlusion or stenosis. The guidewire or an occlusion-penetrating tool may then be passed through the occlusion or stenosis. However, traversing the occlusion may create enough backward force in the guide catheter to dislodge the guide catheter from the ostium of the artery being treated. This can make it difficult or impossible for the interventional cardiologist to treat the occlusion or stenosis.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates a commercially available catheter with a support balloon deployed downstream in a vessel with a chronic total occlusion;

FIG. 2 illustrates an example of a conventional support catheter with an inflatable balloon that has been positioned to support a guidewire as a user attempts to penetrate an occlusion in an artery having a tortuosity;

FIG. 3 illustrates another example of a conventional support catheter with an inflatable balloon that has been positioned to support a guidewire as a user attempts to penetrate an occlusion in an artery having a tortuosity;

FIG. 4A illustrates the trajectory of a guide catheter as it is kicked back out of the ostium when pressure is applied to the guidewire;

FIG. 4B illustrates how a guide catheter can be displaced from the ostium even when a guide catheter extension is used, such as a Guideliner™;

FIG. 5A illustrates an example of an extension member for a guide catheter, the extension member having an expandable support balloon configured to support the extension member and prevent dislodgement from backwards pressure during penetration of the occlusion, shown with the support balloon in a deflated state;

FIG. 5B illustrates the extension member of FIG. 5A, shown with the support balloon in an inflated state;

FIG. 5C shows a close-up view of several portions of the extension member,

FIGS. 5D shows a cross-sectional view of one example of an elongated pushing member and inflation lumen;

FIGS. 5E shows a cross-sectional view of a second example of an elongated pushing member and inflation lumen;

FIGS. 5F shows a cross-sectional view of a third example of an elongated pushing member and inflation lumen;

FIGS. 5G shows a cross-sectional view of a fourth example of an elongated pushing member and inflation lumen; and

FIG. 6 illustrates the extension member of FIGS. 5A and 5B, after having been positioned in an ostium with the support balloon inflated adjacent an occlusion, all in accordance with various embodiments.

### DETAILED DESCRIPTION OF DISCLOSED EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “NB” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Disclosed in various embodiments are extension members for guide catheters that may be used to navigate a tortuosity, such as navigating into an ostium of a coronary artery. In various embodiments, once the extension member has been positioned deep in the ostium, a support balloon may be deployed near the distal tip of the extension member in order to provide axial support to the extension member so that it may be retained in position even when backwards (e.g., proximal-ward) pressure is generated by a user attempting to traverse a stenosis or occlusion distal to the extension member.

In various embodiments, the extension member may take the form of an elongated, metal-reinforced tubular portion having greater stiffness than a conventional guide catheter, an elongated pushing member that is substantially non-compressible in an axial direction, and an expandable support balloon coupled to the reinforced tubular portion adjacent the distal tip of the extension member. In various embodiments, the expandable support balloon may encircle the reinforced tubular portion of the extension member, and it may be positioned so that when it is fully inflated, the distal edge of the expandable support balloon may be no more than 1 mm proximal to the distal tip of the extension member. In various embodiments, the expandable support balloon may have in inflated length of no more than 4 mm, and inflated diameter of between 1 mm and 6 mm, and it may be sufficiently compliant so as to fully inflate with a pressure of 0.5-1.0 atmospheres, thus providing an atraumatic contact with the vessel walls.

Prior to the present disclosure, attempts to provide support to the guide catheter to prevent backward dislodgement (such support referred to herein as “backup support”) from the ostium of a coronary artery have not been fully successful. Some have used guiding catheters that, through a combination of shape and stiffness, are configured to draw backup support from engaging the wall of the aortic arch opposing the ostium of the artery that is being accessed. Often, a guide catheter that is stiff enough to provide adequate backup support may be too stiff to be safely inserted without the possibility of causing damage to the arterial wall. In addition, some have attempted to “deep seat” the guide catheter, but the rigidity of the catheter risks damaging the artery wall or occluding the artery. As used herein, the term “deep seating” refers to inserting the catheter more deeply into the ostium of the coronary artery than typically is done with a conventional guide catheter.

Others have used guide catheters that include a retractable appendage to engage the opposing wall of the aorta or to stiffen a bend in the catheter to provide backup support. These guide catheters tend to be mechanically complex and have not been widely adopted. Another approach has been to use a guide catheter having an expandable balloon near the distal end. These devices have been difficult to navigate around a tortuosity to position the device near an occlusion. Still others have used a smaller guide catheter within a larger guide catheter in order to provide added support for the traversing of stenoses or occlusions, for instance to deep seat the guide catheter within the ostium of the arterial branch. However, deep seating with a commonly available guide catheter creates the risk that the relatively stiff, fixed curve, guide catheter will damage the artery. This damage may lead to dissection of the artery when the catheter is advanced past the ostium. The very compliant balloon of the present disclosure is less likely to dissect the coronary artery.

By contrast, the present disclosure enables the use of an extension member having an expandable support balloon that for a guide catheter that may allow the extension to be deep seated in the ostium and still provide sufficient backup support to allow a variety of tools to be used to penetrate and traverse a chronic total occlusion. Existing extension devices, such as the Guideliner™ from Vascular Solutions in Minneapolis, Minn., may be used to more closely approach a stenosis or occlusion in a coronary artery, but such devices still may not afford sufficient purchase to allow a densely fibrous occlusion to be traversed. Furthermore, once a guidewire has been passed through an occlusion, it is still necessary to pass a stent or balloon device in order to widen the lumen of the vessel at the blockage. Such procedures produce high levels of back pressure, and even a reinforced extension member may “bounce back” out of the ostium when sufficient pressure is applied.

Thus, the disclosed extension members include a selectively inflatable expandable support balloon adjacent the distal tip of a flexible, reinforced extension member, which balloon may be inflated to engage the walls of the coronary artery once the extension member has been deep seated in the ostium. In various embodiments, the balloon is sized and positioned to allow the extension member to be used to navigate one or more tortuosities, while still providing sufficient backup support. Inflation of the expandable balloon provides the additional purchase needed to be able to advance a stent or balloon catheter into and through a dense or fibrous occlusion.

The disclosed extension members may have a metal-reinforced tubular portion that is between about 20 cm and 35 cm long and an elongated pushing member that is approximately 90-120 cm long so that it may extend beyond the proximal end of a conventional guide catheter. In various embodiments, the reinforced tubular portion may have an outer diameter that is sized so that the largest diameter of the reinforced tubular portion (including the collapsed support balloon, mounted on an exterior surface thereof), may pass through the inner lumen of a conventional guide catheter (for example, such as a standard 5 French-8 French guide catheter) without damaging the support balloon. In some embodiments, the extension member may be adapted to run over a standard 0.014 inch coronary guidewire, whereas in other embodiments, it may be adapted to use a monorail delivery system. In various embodiments, the extension member may be delivered through existing hemostatic valves that are commonly used with guide catheters, while still allowing injections through an existing Y adapter. In addition, in various embodiments, the reinforced tubular portion of the extension member may have a hollow lumen with an inner diameter that is appropriately sized for delivering standard coronary treatment devices such as stents and balloon catheters.

In some embodiments, the metal-reinforced tubular portion of the extension member may have an outer diameter (e.g., including the outer diameter of the collapsed support balloon) that is smaller than the internal diameter of an 8 French, 7 French, or 6 French guide catheters, such as those commonly used in interventional cardiology procedures. For reference, an 8 French catheter has an internal diameter greater than or equal to 0.088 inches; a 7 French catheter has an internal diameter greater than or equal to 0.078 inches; and a 6 French guide catheter has an internal diameter greater than or equal to 0.070 inches. In various embodiments, the outer diameter of the reinforced tubular portion of an extension member as disclosed herein may have an outer diameter (e.g., including the diameter of the collapsed support balloon) of less than about 0.085 inches when used with an 8 French guide catheter; less than about 0.075 inches when used with a 7 French guide catheter; or less than about 0.062 inches when used with a 6 French guide catheter.

In some embodiments, the metal-reinforced tubular portion may be provided with a shallow recess adjacent the tip that accommodates the thickness of the collapsed balloon. In some embodiments, this recess may be created by providing the reinforced tubular member with a thinner wall portion in this region. In some embodiments, thinning the wall of the tube in this region may render the distal tip portion of the reinforced tubular portion more flexible as compared to the rest of the reinforced tubular portion.

In various embodiments, this narrow exterior profile (e.g., despite the presence of the balloon), makes the device optimal to allow treatment of chronic total occlusions. For instance, in some embodiments, after the occlusion is partially or completely traversed with a guidewire, the ample lumen of the disclosed extension member allows a balloon or stent to be passed through the device and advanced to or through the occlusion. With the compliant balloon inflated, the extension member is less likely to “snap back” under pressure. Additionally, when crossing a stenosis, the compliant nature of the balloon makes it less likely that the coronary artery will be torn or that the vessel will be traumatized.

In some embodiments, the reinforced tubular portion of the extension member may include regions having different degrees of flexibility, such as a distal, more flexible portion and a proximal, more rigid portion. In various embodiments, the distal, more flexible portion may include metallic fibers in a braided or coiled pattern, and in specific, non-limiting examples, it may be lined with a PTFE liner and covered on its exterior with Pebax®. Although the reinforced tubular portion of the extension member is generally about 20-35 cm long, it may be longer or shorter depending on the specific patient and application. In some embodiments, the reinforced tubular portion may include three or more portions, each having its own degree of flexibility. In general, the flexibility of each portion may decrease in flexibility as one moves from distal to proximal along the reinforced tubular portion.

In some embodiments, the most proximal, more rigid portion of the reinforced tubular portion may be formed from a stainless steel or Nitinol tube. In some embodiments, the proximal, substantially rigid portion may be joined to the distal, more flexible portion(s) by welding. In various embodiments, the most proximal, more rigid portion may include a cutout portion and a full circumference portion. For example, the cutout portion may include a section where about 45%-90% of the circumference of the tubular structure has been removed, leaving an arcuate member. In various embodiments, this arcuate member may extend proximally 90-120 cm to form an elongated pushing member. In some embodiments, the elongated pushing member may include a plurality of radially oriented slits or other cuts to increase and control the flexibility of the most rigid portion.

In operation, the extension member may be inserted into a standard guide catheter that has been placed into a blood vessel that communicates with the aorta and advanced to the ostium of a coronary artery, and the extension member and guide catheter may be threaded over a preplaced 0.014 inch guidewire or along a monorail and advanced up the aorta until the distal end of the guide catheter is passed into the ostium of the coronary artery. Once the guide catheter has been placed in the ostium, the extension member may be advanced beyond the distal tip of the guide catheter until it has been inserted sufficiently into the ostium of the coronary artery to achieve deep seating. During this entire process, at least a portion (e.g., a proximal portion) of the reinforced tubular portion of the extension member may remain inside of the guide catheter.

Following deployment of the extension member in the desired location, the expandable support balloon may be inflated (e.g., with saline, air, or a dye solution) fully or partially using a pressure of no more than 0.05-1.0 atmospheres, until it makes contact with the walls of the coronary artery. In various embodiments, the balloon may be inflated via a slender inflation lumen running the length of the device, generally parallel to the longitudinal axis of the device. In some embodiments, the inflation lumen may be disposed within the wall of the tubular extension member, and may also be disposed in or along the elongated pushing portion. Once the support balloon has been inflated to anchor the distal tip of the extension member in the coronary artery, a cardiac treatment device, such as a guidewire, balloon, or stent, may be passed through the guide catheter and extension member and into the coronary artery. As described below, the deployment of the support balloon on the extension member provides sufficient backup support to prevent the guide catheter and/or extension member from becoming dislodged from the ostium of the coronary artery while directing the coronary therapeutic device past a tough lesion such as a stenosis or a chronic arterial occlusion.

As explained above, a guide catheter inserted into the ostium of a branch artery where it branches off from a larger artery is subject to force vectors that tend to dislodge the distal end of the guide catheter from the ostium of the branch artery when a user attempts to direct a guidewire or other interventional cardiology device past an occlusive or stenotic lesion in the branch artery. Although the discussion below refers to a guide wire, it is to be understood that similar principles apply to other interventional cardiology devices including balloon catheters and stents.

One of the forces that acts on the guide catheter is an axial force substantially along the axis of the branch artery and the portion of the guide catheter that is seated in the ostium. This force vector is a reactive force created by the pushing back of the guide wire against the guide catheter as the user tries to force the guidewire through or past the lesion. It tends to push the distal end of the catheter out of the ostium in a direction parallel to the axis of the branch artery and the axis of the distal end of the guide catheter. Another of the force vectors that acts on the guide catheter is a shearing force that tends to dislodge the distal end of the guide catheter from the ostium of the branch artery in a direction perpendicular to the axis of the branch artery and the axis of the distal end of the guide catheter. This force vector arises from curvature of the guide catheter near its distal end and the guidewire pushing on the curved portion of the guide catheter as the user applies force to the guidewire. The extension members having support balloons described herein may assist in resisting both the axial forces and the shearing forces that tend to dislodge a guide catheter from the ostium of a branch artery.

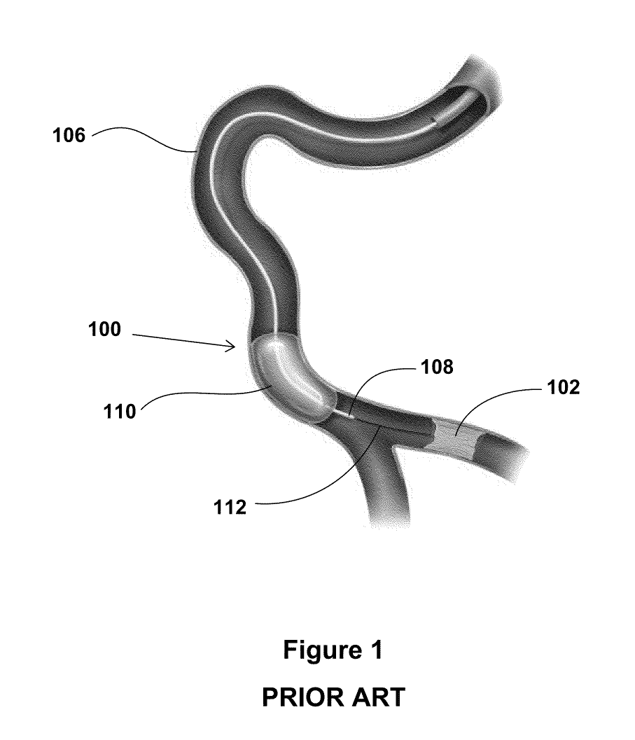

FIG. 1 illustrates a commercially available support catheter having a support balloon deployed downstream in a vessel with a chronic total occlusion. As illustrated, a the support catheter 100 has been placed downstream in a vessel 106 that takes a relatively straight path, which permits navigation of the distal tip 108 of the support catheter 100 close to the occlusion 102. The support balloon 110 has an elongated shape that provides axial support when the guidewire 112 is used to penetrate the occlusion 102. However, the support balloon 110 is too long to be able to navigate and position the device around many tortuosities.

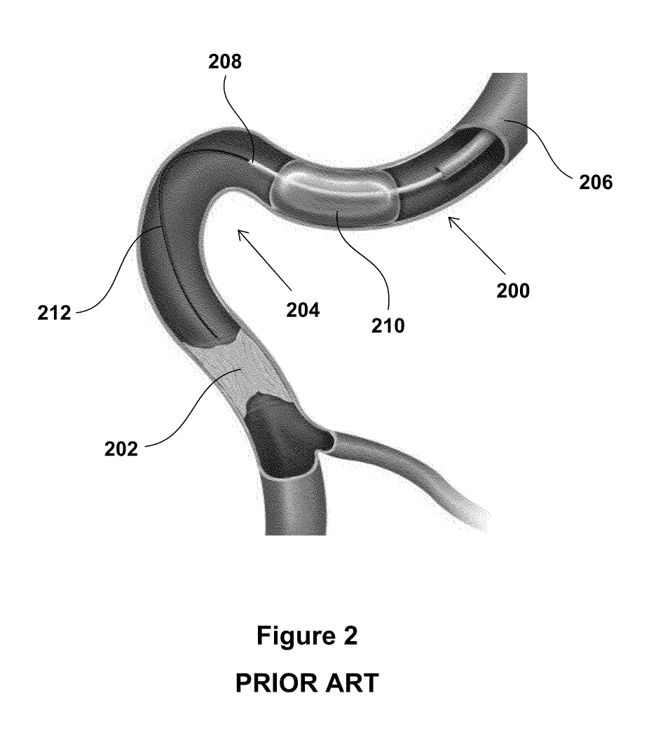

FIG. 2 illustrates an example of a conventional support catheter with an inflatable balloon that has been positioned in an artery having a tortuosity as a user attempts to penetrate an occlusion, in accordance with various embodiments. As can be seen from FIG. 2, the support catheter 200 cannot be placed close to the occlusion 202 because of a tortuosity 204 in the artery 206 that prevents navigation of the distal tip 208 of the support catheter 200 close to the occlusion 202. Additionally, the support balloon 210 has an elongated shape that prevents installation in or near a tortuosity 204. As a result, the guidewire 212 does not have enough purchase to penetrate the occlusion 202.

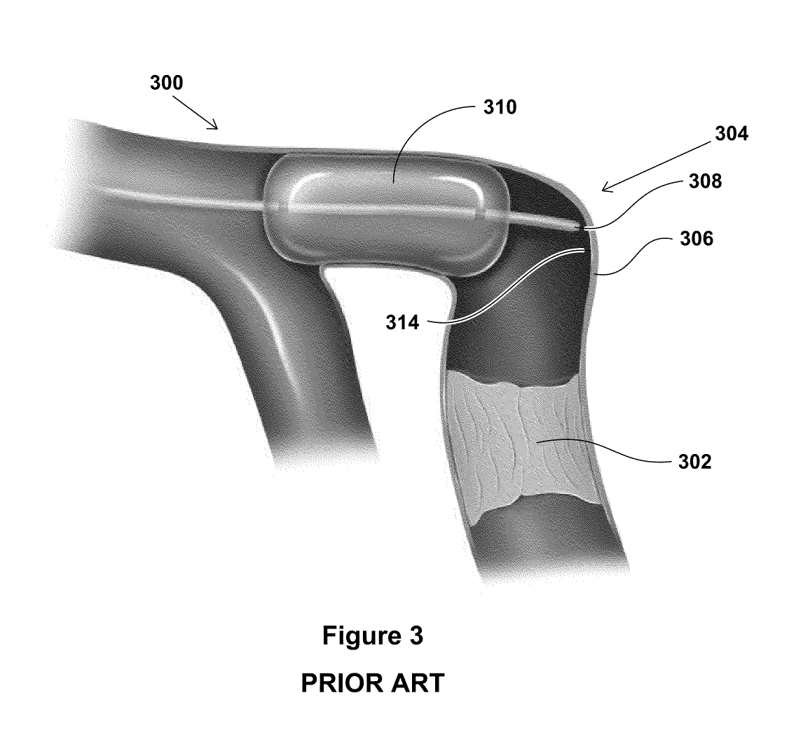

FIG. 3 illustrates another example of a conventional support catheter with an inflatable balloon that has been positioned to support a guidewire as a user attempts to penetrate an occlusion in an artery having a tortuosity, in accordance with various embodiments. Similar to the scenario depicted in FIG. 2, FIG. 3 illustrates a support catheter 300 that cannot be placed close to an occlusion 302 because of a tortuosity 304 in the artery 306. In the illustrated embodiment, the tortuosity 304 is too severe to permit navigation of the elongated conventional support balloon 310, and the distal tip 308 of the support catheter 300 abuts the arterial wall 314. Additionally, the positioning of the support balloon 310 far back from the distal tip 308 makes it impossible to navigate the tortuosity 304. As a result, there is no way to maneuver a guidewire close enough to penetrate the occlusion 302.

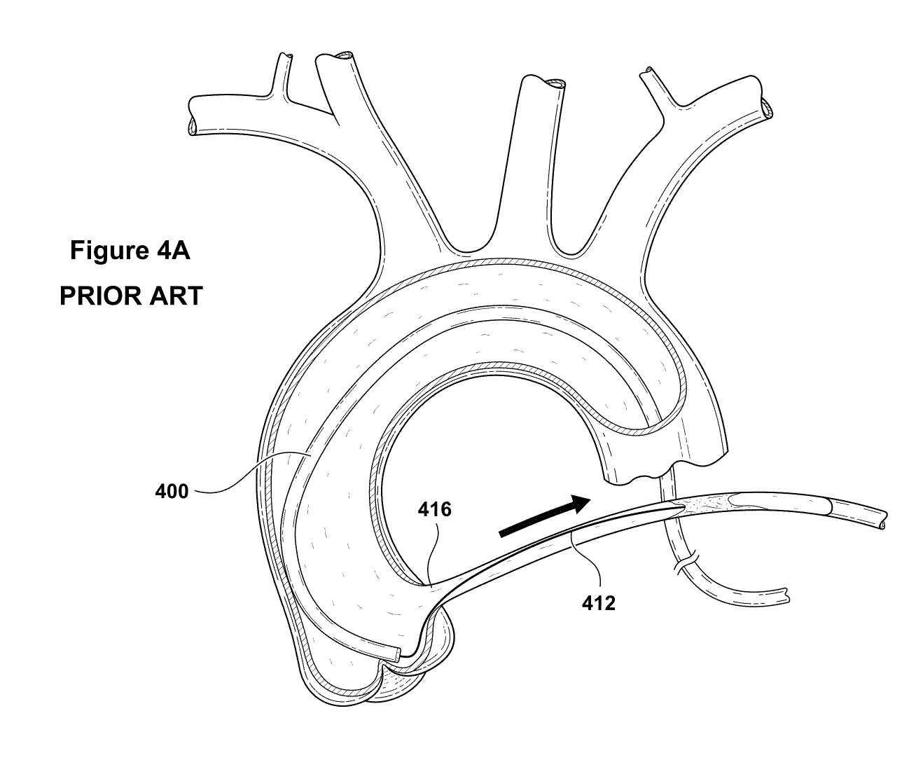

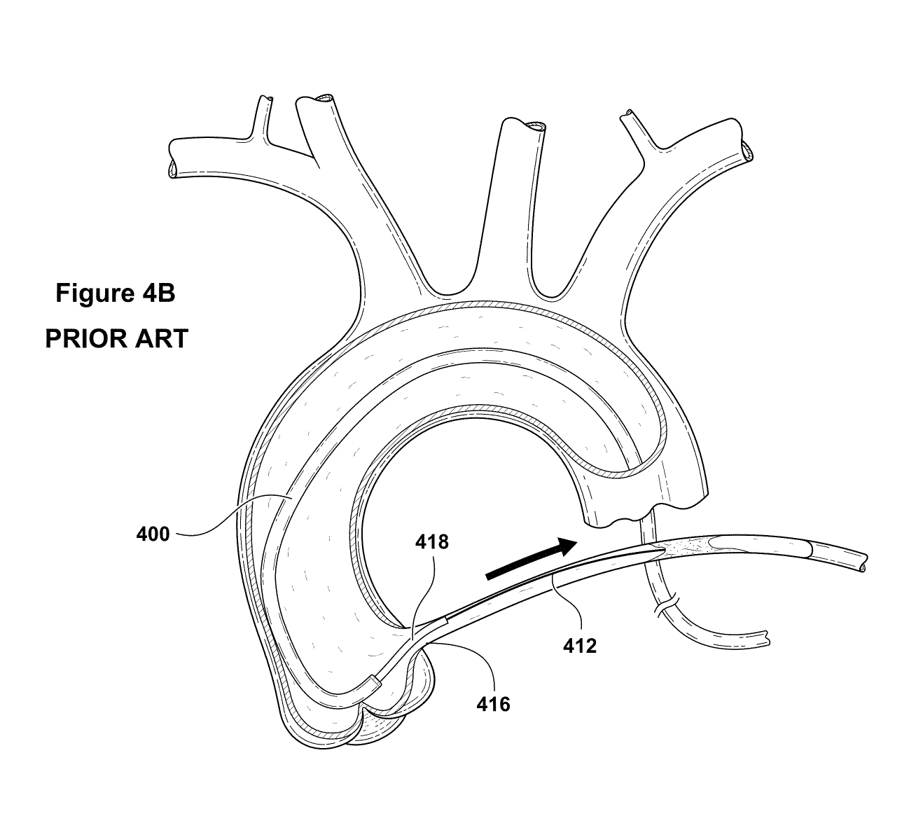

FIG. 4A illustrates the trajectory of a guide catheter as it is kicked back out of the ostium when pressure is applied to the guidewire, in accordance with various embodiments. As illustrated, even when a guide catheter 400 is positioned in an ostium 416, the backwards pressure that is created when a user exerts force on the guidewire 412 can cause the guide catheter 400 to back out of the ostium 416, making it impossible to perform the procedure. Similarly, FIG. 4B illustrates how a guide catheter can be displaced from the ostium even when a guide catheter extension is used, such as a Guideliner™, in accordance with various embodiments. As illustrated, even when a support catheter 400 is positioned in an ostium 416 with the aid of a catheter extension device 418, the backwards pressure that is created when a user exerts force on the guidewire 412 can still cause the guide catheter 400 and extension device 418 to back out of the ostium, making it impossible to perform the procedure.

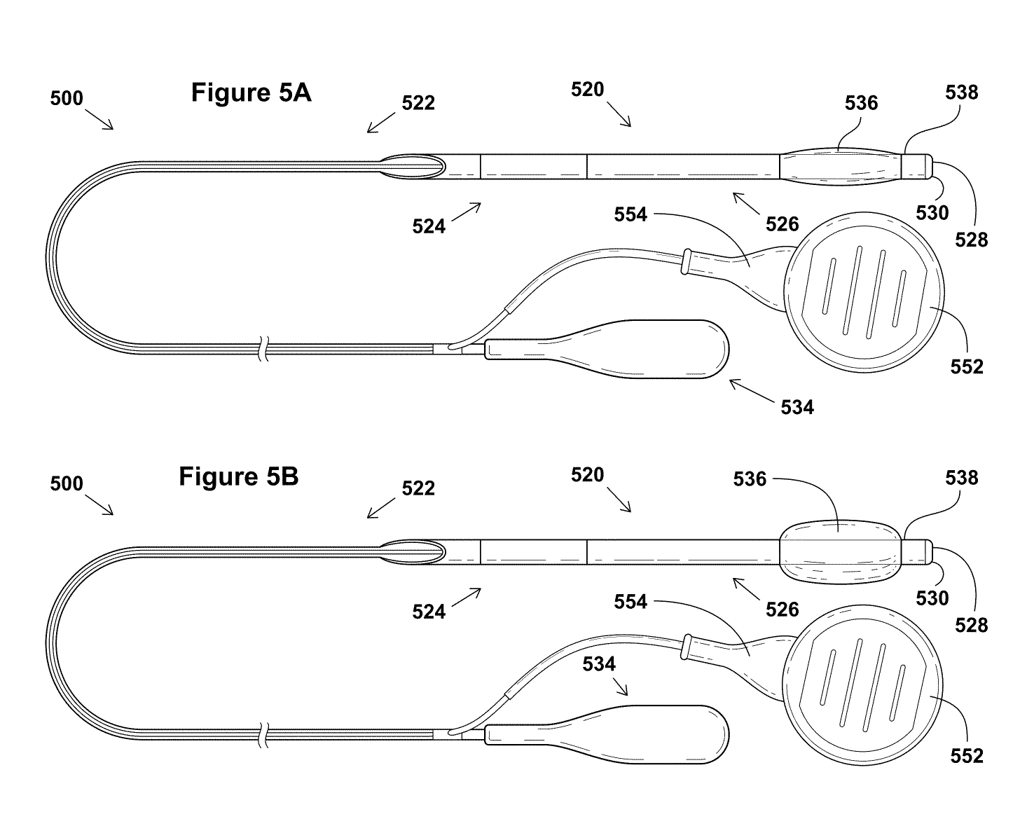

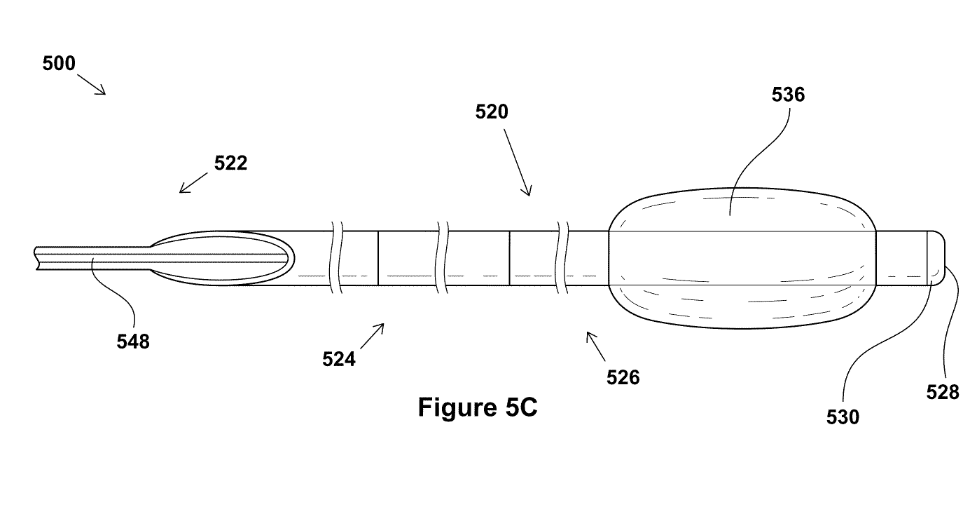

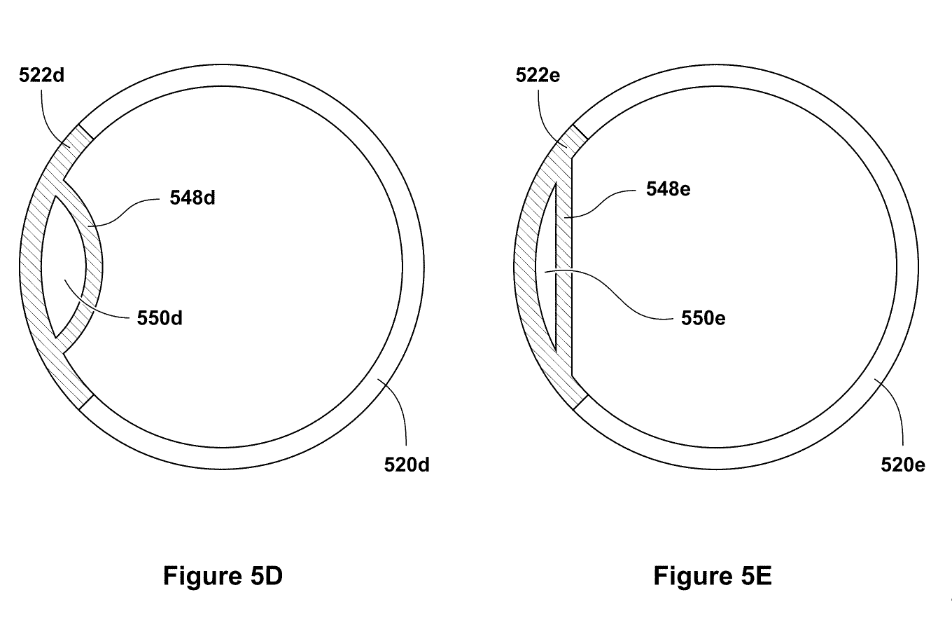

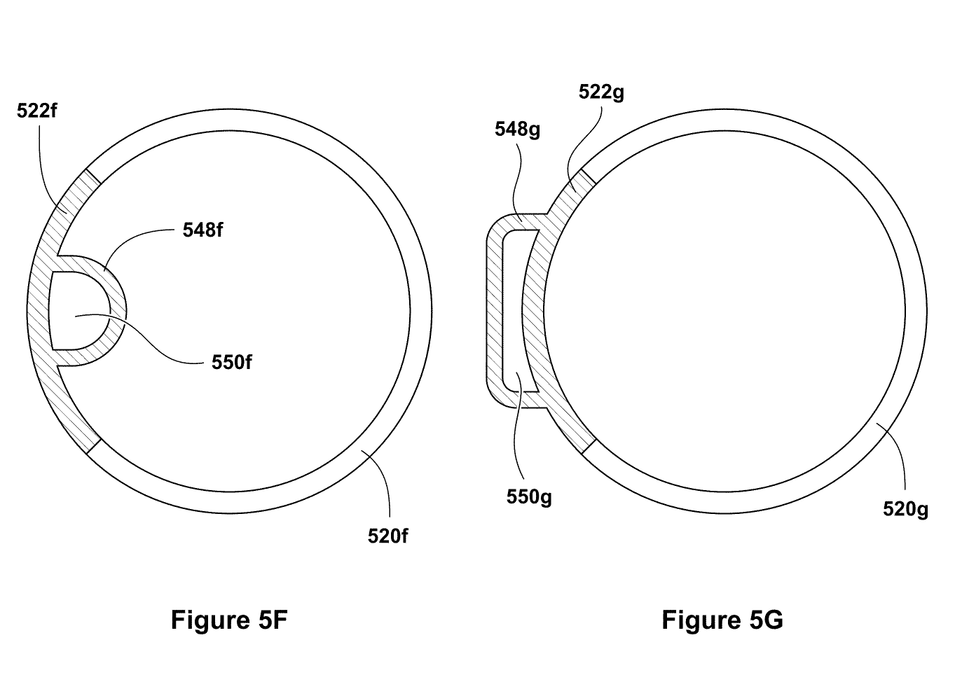

FIG. 5A illustrates an example of an extension member for a guide catheter, the extension member having an expandable support balloon configured to support the extension member and prevent dislodgement from backwards pressure during penetration of the occlusion, shown with the support balloon in a deflated state. FIG. 5B illustrates the extension member of FIG. 5A, shown with the support balloon in an inflated state, both in accordance with various embodiments. Referring to FIGS. 5A and 5B, the extension members 500 of the present disclosure generally include a reinforced tubular portion 520 and an elongated pushing member 522. In various embodiments, reinforced tubular portion 520 may include different regions, such as a distal, more flexible portion 524 and a proximal, more rigid portion 526. The overall length of the extension member 500 typically may be approximately 125 cm, however this length should not be considered limiting. In various embodiments, the distal end of the extension member may include a bump tip 528 and a marker band 530.

In various embodiments, bump tip 528 may be relatively flexible, may also be slightly tapered, and may formed, for example, from 4033 Pebax®. In various embodiments, bump tip 528 generally is not longer than 1 mm in length (measuring from distal to proximal), and may be about 0.1 mm in length in some embodiments. In some embodiments, marker band 530 may be formed of a radiopaque material such as platinum/iridium alloy usually at a 90/10 ratio, and may be sandwiched between an outer Pebax® material and a PTFE liner.

In various embodiments, the reinforced tubular portion 520 may include a flexible membrane disposed immediately adjacent the bump tip 528. In various embodiments, this flexible membrane forms an expandable support balloon 536, which is secured to the reinforced tubular portion 520. In various embodiments, the support balloon 536 may be selectively expanded (see FIG. 5B) and collapsed (see FIG. 5A), and when in the expanded state, the distal end 538 of the support balloon 536 may be positioned at a distance D from the distal (bump) tip 528. In some embodiments, the distal end 538 of the support balloon 536 is immediately adjacent the distal end of the extension member 500. In other embodiments, distance D may be between about 0.1 cm and 1.0 cm from the distal end of extension member 500. In various embodiments, when so positioned, the support balloon 536 may be free from interference by sharp implements exiting the lumen of the extension member 500, while also allowing the support balloon 536 to be positioned very close to the occlusion (e.g., having a distance of as little as 0.1 cm-1.0 cm, if desired, in order to provide maximum backup support. In some embodiments, the support balloon 536 may comprise or be formed entirely from nylon.

In various embodiments, the support balloon 536 may have an interior chamber that may vary in volume when expanded and contracted (see, e.g., FIGS. 5B and 5A, respectively). In some embodiments, a narrow-diameter catheter or lumen may be provided that extends the length of extension member 500 and communicates with the interior chamber of the support balloon 536. In some embodiments, this narrow-diameter catheter may be used to pump a fluid, such as saline, air, or a dye solution, into and out of the support balloon 536, thus inflating and deflating the support balloon 536 as desired. In some embodiments, the narrow-diameter catheter may run along or within the length of the elongated pushing member 522, wherein in other embodiments, the elongated pushing member may include a hollow bore through which the fluid may be directed (better seen in FIG. 5C, which is discussed below).

In various embodiments, the support balloon 536 may be very compliant, and may inflate with a very slight change in pressure, such as between 0.5 and 1.0 atmospheres. In some embodiments, the support balloon 536 may have an expanded length (measured from distal to proximal) of about 4 mm, and an expanded diameter of about 1-6 mm.

In various embodiments, the reinforced tubular portion 520 may be reinforced with braid or coil reinforcement, for instance formed of metal, plastic, graphite, or composite structures. In various embodiments, reinforced tubular portion 520 may be lined on the interior by a PTFE liner and covered on the exterior by a Pebax® material. In some embodiments, the reinforced tubular portion 520 is between 20 cm and 35 cm in length.

In various embodiments, the more rigid portion 526 of the reinforced tubular portion 520 may be secured to the more flexible portion 524 by, for example, welding or bonding. In various embodiments, the more rigid portion 526 may be formed from a hypotube or a section of stainless steel or Nitinol tubing, although other substantially rigid materials may be used as well. In various embodiments, the more rigid portion 526 may include a cutout portion and a short full circumference portion. For example, the cutout portion may include a section where about 45%-90% of the circumference of the tubular structure has been removed, leaving an arcuate member. In various embodiments, this arcuate member may extend proximally 90-120 cm to form the elongated pushing member 522. In some embodiments, the elongated pushing member 522 may include a plurality of radially oriented slits or other cuts to increase and control the flexibility of the more rigid portion 526.

In various embodiments, extension member 500 may include a variety of regions having different levels of flexibility. For example, starting at its distal end, a first portion may have a flexural modulus of about 13,000 PSI plus or minus 5000 PSI; a second, more proximal portion may have a flexural modulus of about 29,000 PSI plus or minus 10,000 PSI; a third, even more proximal portion may have a flexural modulus of about 49,000 PSI plus or minus 10,000 PSI; and a fourth, still more proximal portion may have a flexural modulus of about 107,000 PSI plus or minus 20,000 PSI.

In various embodiments, a grip portion 534 may be provided that, in some embodiments may include gripping ears that may extend outwardly from grip portion 534 substantially radially and be shaped for convenient gripping by a user. Adjacent the grip portion 534 is an inflation device 552 and control valve 554 that may be used to inflate the support balloon 536 in some embodiments.

FIG. 5C shows a close-up view of several portions of the extension member 500, including the elongated pushing member 522 and inflation catheter 548, the tubular portion 520, including the distal, more flexible portion 524 and the proximal, more rigid portion 526, the expandable support balloon 536, the marker band 530, and the bump tip 528. As can be seen from the cross sectional views of FIGS. 5D-5G, the interrelationships between the contours of the tubular portion 520, 520d, 520e, 520f, 520g, elongated pushing member 522, 522d, 522e, 522f, 522g, inflation catheter 548, 548d, 548e, 548f, 548g, and inflation catheter lumen 550d, 550e, 550f, 550g, may take any of several forms, including an embodiment illustrated in FIG. 5D, wherein the inflation catheter 548d extends from the elongated pushing member 522d in an arcuate shape, creating a lumen 550d having two curved sides when viewed in cross section. In the embodiment illustrated in FIG. 5E, the inflation catheter 548e extends from the elongated pushing member 522e as a straight wall, creating a lumen 550e having a semicircular cross section. In the embodiment illustrated in FIG. 5f, the inflation catheter 548f extends from the elongated pushing member 522f as a semicircular wall, creating lumen 550f. And, in the embodiment illustrated in FIG. 5G, the inflation catheter 548g extends from the outside curve of the elongated pushing member 522g, creating lumen 550g. In each of FIGS. 5D-5G, the tubular portion 520d, 520e, 520f, 520g may be seen extending behind the illustrated plane.

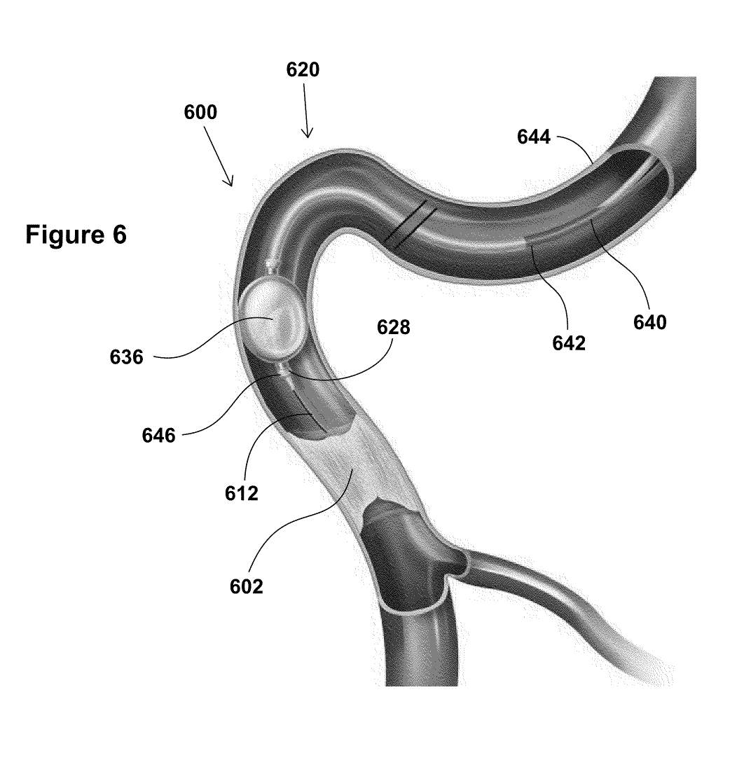

FIG. 6 illustrates the extension member for a guide catheter of FIGS. 5A and 5B, after having been positioned in an ostium with the support balloon inflated adjacent an occlusion, in accordance with various embodiments. In operation, a guide catheter 640 may be inserted into a major blood vessel in the body, such as the aortic arch, over a guidewire 612 (or monorail system, in some embodiments) and the distal end 642 of the guide catheter 640 may be brought into proximity of an ostium of a smaller branch blood vessel, such as a coronary artery 644. The extension member 600 may then be inserted through guide catheter 640 and over guidewire 612. Guide catheter 640, guidewire 612, and extension member 600 may then be manipulated to insert and the distal end 646 of the extension member 600 into the ostium of the blood vessel that branches off from the major blood vessel. The bump tip 628 of extension member 600 may be inserted well into the ostium of the coronary artery 644 or other blood vessel until it achieves a deep seated position.

Once placed, support balloon 636 may then be inflated in order to anchor the extension member 600 firmly in place in order to provide backup support for any desired occlusion- or stenosis-traversing procedures.

An interventional cardiology treatment device such as a guidewire, catheter bearing a stent, or a balloon may then be inserted through the lumen of the extension member 600, a portion of which remains inside guide catheter 640. When the interventional cardiology device reaches a stenosis or occlusion 602 in the coronary artery 644 or another branch blood vessel, force may be applied to the interventional cardiology device while the extension member 600 provide backup support. Thus, the back force that would tend to dislodge the extension member 600 from a deep seated position in the ostium in the branch blood vessel may be transferred to the support balloon 636, the reinforced tubular portion 620, and the elongated pushing member (not shown). During use, in some embodiments, a user may apply a force to the proximal end of the extension member 600 to resist dislodging of the extension member 600 from the ostium of the branch artery. Once the procedure has been completed, the support balloon 636 may be deflated, and the extension member 600 may be withdrawn from the body.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. An extension member for use with a standard guide catheter, the guide catheter having a proximal end, a distal end, and a first central lumen interconnecting the proximal and distal ends, the extension member comprising:
a tubular structure having a proximal end, a distal end, a second central lumen interconnecting the proximal and distal ends, and comprising a reinforcing element spanning a circumference of the tubular structure and extending from the proximal end to the distal end,
an elongated pushing member coupled to the proximal end of the tubular structure such that when so coupled, the length of the extension member is greater than 100 cm, the tubular structure and elongated pushing member being substantially non-compressible in an axial direction;
an expandable balloon having an expanded state and a compressed state, wherein the expandable balloon is disposed on an outside surface of the tubular structure and positioned immediately adjacent to the distal end of the tubular structure, and wherein the expandable balloon, when inflated, has a substantially cylindrical shape; and
an inflation device in fluid communication with the expandable balloon and selectively operable to expand and collapse the expandable balloon, wherein the tubular structure has an outer diameter sized to be insertable, together with the collapsed balloon, into the first central lumen.

2. The extension member of claim 1, wherein the tubular structure includes a distal portion adapted to be extended beyond the distal end of the guide catheter while a proximal portion remains within the lumen of the guide catheter, such that the support member is positionable to assist in resisting axial and shear forces when the distal end of the guide catheter is inserted into an ostium of a coronary artery.

3. The extension member of claim 2, wherein expansion of the expandable balloon assists in resisting axial and shear forces when the distal end of the guide catheter is inserted into an ostium of a coronary artery.

4. The extension member of claim 1, wherein the expandable balloon has a length of no more than 4 mm in the proximal-distal direction when in the expanded state.

5. The extension member of claim 1, wherein the expandable balloon has a diameter of no more than 6 mm when in the expanded state.

6. The extension member of claim 4, wherein the expandable balloon is sufficiently compliant to reach the expanded state with the application of no more than 1.0 atmospheres of pressure from the inflation device.

7. The extension member of claim 6, wherein the expandable balloon is sufficiently compliant to reach the expanded state with the application of no more than 0.5 atmospheres of pressure from the inflation device.

8. The extension member of claim 1, wherein the expandable balloon comprises nylon.

9. The extension member of claim 7, wherein the fluid communication between the expandable balloon and the inflation device comprises a slender catheter that extends along and couples to the full length of the elongated pushing member.

10. The extension member of claim 7, wherein the fluid communication between the expandable balloon and the inflation device comprises a lumen formed within and extending along the full length of the elongated pushing member.

11. The extension member of claim 1, wherein the reinforcing element comprises metallic elements in a braided or coiled pattern.

12. The extension member of claim 11, wherein the reinforcing element comprises NITINOL® or steel.

13. The extension member of claim 1, wherein the proximal end of the tubular structure comprises a substantially rigid portion.

14. The extension member of claim 1, wherein the elongated pushing member is substantially arcuate in cross section.

15. The extension member of claim 14, wherein the elongated pushing member comprises a plurality of radially-oriented cuts.

16. The extension member of claim 15, wherein the radially-oriented cuts increase flexibility of the elongated pushing member.

17. A method of using the extension member of claim 1, the method comprising:
inserting the standard guide catheter over a guidewire and into a femoral artery;
advancing the standard guide catheter through the vasculature to an ostium of a coronary artery using the elongated pushing member;
advancing the extension member through the standard guide catheter through the distal tip of the standard guide catheter;
using the guide catheter, guide wire, and elongated pushing member to advance the extension member out of the standard guide catheter until the distal end of the extension member is deeply seated in the ostium, the proximal end of the tubular structure remaining in the standard guide catheter;
inflating the expandable balloon sufficiently to contact a wall of the coronary artery;
using an occlusion-penetrating tool to traverse an occlusion or stenosis in the coronary artery;
collapsing the expandable balloon; and
withdrawing the extension member through the femoral artery.

18. The method of claim 17, wherein inflating the expandable balloon comprises operating the inflation device to apply a pressure of no more than 1.0 atmospheres.

19. The method of claim 18, wherein inflating the expandable balloon comprises operating the inflation device to apply a pressure of no more than 0.5 atmospheres.

20. The method of claim 17, wherein using the guide catheter, guide wire, and elongated pushing member to advance the extension member out of the standard guide catheter comprises advancing the extension member until the distal tip of the extension member closely apposes an occlusion or a stenosis.
